(19) United States
(12) Patent Application Publication
(10) Pub. No.: US 2026/0325365 A1
(43) Pub. Date: Oct. 22, 2026

(54) WEARABLE CAPACITANCE-MEASURING DEVICE WITH MOBILE APPLICATION INTEGRATION

(71) Applicant: vdW Design, LLC, Madison, WI (US)

(72) Inventors: Daniel W. VAN DER WEIDE, Madison, WI (US); Hudson CZUPRYNA, Hudson, WI (US)

(21) Appl. No.: 19/653,764

(22) Filed: Apr. 21, 2026

Related U.S. Application Data
(60) Provisional application No. 63/792,090, filed on Apr. 21, 2025.

(51) Int. Cl. A61F 13/42; A61F 13/84; H01Q 1/22

(52) U.S. Cl. CPC A61F 13/42; A61F 13/84; H01Q 1/22; A61F 2013/424; A61F 2013/8488

(57) ABSTRACT

A wearable capacitance-measuring device with mobile application integration is disclosed. The device comprises a flexible substrate that can be worn or incorporated into personal care products, a capacitance-sensing element made of conductive wire or ink patterns embedded in the substrate, and a Near Field Communication (NFC) chip connected to the capacitance-sensing element. The NFC chip measures changes in capacitance in response to environmental factors such as moisture or pressure, stores the measurement data, and communicates with an NFC-enabled smartphone when scanned. A mobile application retrieves and interprets the capacitance measurements, providing real-time information to users without requiring batteries or active power sources in the wearable device. Example applications include smart diapers for moisture detection, health monitoring wearables, and environmental sensing products, all offering passive operation and on-demand data access through a simple scanning interaction.

### CROSS REFERENCE TO RELATED APPLICATION

The present application claims the priority benefit of U.S. Provisional Patent Application No. 63/792,090, filed on Apr. 21, 2025, which is incorporated herein by reference in its entirety.

### TECHNICAL FIELD

The present disclosure relates generally to wearable technology and sensor systems, and more particularly to passive capacitance-measuring devices integrated with near-field communication (NFC) technology that interact with mobile applications to provide real-time measurement data in applications such as health monitoring, moisture detection, and environmental sensing.

### BACKGROUND

Effective monitoring of environmental conditions through wearable technology remains a significant challenge in various healthcare, wellness, and personal care applications. Traditional wearable sensing devices often rely on active electronic components, power-hungry sensors, or complex circuitry to detect and communicate changes in environmental parameters such as moisture, pressure, or temperature. These conventional approaches typically require batteries or other power sources, which introduce limitations related to size, weight, cost, and maintenance requirements.

Current solutions for applications like moisture detection in diapers, for example, often involve bulky sensor packages that are impractical for everyday use, expensive to manufacture, and create waste disposal concerns. Some existing smart diaper systems incorporate disposable electronic sensors with separate receiver units, increasing both complexity and cost while reducing user convenience. Alternative approaches using color-changing indicators lack the ability to provide digital data or remote notifications, limiting their utility in modern caregiving scenarios.

In health monitoring applications, many wearable devices require continuous power to operate sensors and communication modules, necessitating regular recharging or battery replacement. This power requirement limits the potential for ultra-thin, lightweight, or disposable monitoring solutions. Furthermore, existing systems typically demand significant user interaction for setup, activation, and data retrieval, creating barriers to adoption for elderly users or time-constrained caregivers.

Near Field Communication (NFC) technology has enabled passive data exchange in applications such as contactless payments and access control, but its potential for environmental sensing in wearable applications remains largely unexplored. While some NFC-enabled sensors exist, they often focus on specific applications without providing a flexible platform that can be adapted across multiple use cases. Additionally, many current solutions fail to effectively integrate the physical sensing element with mobile application interfaces, resulting in fragmented user experiences and limited data utilization.

There is a need for passive, lightweight wearable sensing devices that can measure environmental parameters without batteries, provide data on demand through simple user interactions, and integrate seamlessly with mobile applications for interpretation and notification purposes. Such solutions would be particularly valuable in applications ranging from infant care products to healthcare monitoring and environmental sensing, where simplicity, reliability, and cost-effectiveness are essential requirements.

### SUMMARY

The present disclosure provides, in at least one aspect, wearable devices, comprising a substrate, a capacitance-sensing element on or within the substrate, wherein the capacitance-sensing element changes capacitance in response to environmental conditions, and an NFC component connected to the capacitance-sensing element, configured to measure the capacitance when activated by an external device.

In some embodiments, the environmental conditions comprise at least one of moisture, pressure, or proximity.

In some embodiments, the capacitance-sensing element comprises a conductive material arranged in a pattern.

In some embodiments, the conductive material comprises at least one of conductive ink, conductive wire, conductive thread, or metal foil.

In some embodiments, the pattern comprises interdigitated electrodes.

In some embodiments, the NFC component operates without a battery.

In some embodiments, the substrate is incorporated into at least one of a personal care product, a wearable accessory, or clothing.

In some embodiments, the substrate comprises a biocompatible material.

In some embodiments, the capacitance-sensing element comprises a plurality of sensing zones positioned at different regions of the substrate, wherein each sensing zone is configured to independently measure a localized environmental condition.

In some embodiments, the NFC component comprises an NFC antenna and an integrated circuit comprising a capacitance measurement circuit and onboard memory configured to store at least one measured capacitance value.

The present disclosure provides, in at least one aspect, systems for monitoring environmental conditions, comprising, a wearable device comprising a substrate, a capacitance-sensing element on or within the substrate, and an NFC component connected to the capacitance-sensing element, and a mobile application on a mobile device configured to activate the NFC component, receive capacitance data from the NFC component, and determine a status based on the capacitance data.

In some embodiments, the mobile application is further configured to generate a notification based on the determined status.

In some embodiments, the wearable device operates without a battery.

In some embodiments, the mobile application is further configured to store historical data from a plurality of measurements and display a trend based on the historical data.

In some embodiments, the mobile application is further configured to synchronize the capacitance data with a remote server and provide access to the data from a plurality of authorized user devices.

In some embodiments, the wearable device is incorporated into a personal care product, and wherein the mobile application is configured to interpret the capacitance data to determine a moisture status of the personal care product.

The present disclosure provides, in at least one aspect, methods of measuring environmental conditions, comprising, providing a wearable device comprising a substrate, a capacitance-sensing element on or within the substrate, and an NFC component connected to the capacitance-sensing element, bringing a mobile device into proximity with the wearable device such that the mobile device activates the NFC component via an electromagnetic field, measuring, by the NFC component, a capacitance value of the capacitance-sensing element, transmitting the capacitance value to a mobile application on the mobile device, and determining, by the mobile application, a status based on the capacitance value.

In some embodiments, the method further comprises generating, by the mobile application, a notification based on the determined status.

In some embodiments, the method further comprises storing the capacitance value with a timestamp to create a measurement log, and displaying, by the mobile application, trend information derived from a plurality of stored measurements.

In some embodiments, the method further comprises performing a calibration measurement of the capacitance-sensing element under a known environmental condition, and applying the calibration measurement as a baseline value when determining the status.

Other aspects of the disclosure will become apparent by consideration of the detailed description and accompanying drawings.

### Definitions

The terms “substantially” or “generally” are used to provide flexibility by recognizing that a given characteristic need not be perfectly embodied to have the desired result. Those of ordinary skill in the art will recognize that many characteristics described herein may be essentially present without strict adherence to the characteristic's definition.

The term “coupled,” as used herein, is defined as “connected,” although not necessarily directly, and not necessarily mechanically. The term coupled is to be understood to mean physically, magnetically, chemically, fluidly, electrically, or otherwise coupled, connected or linked and does not exclude the presence of intermediate elements between the coupled elements absent specific contrary language.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “subject,” “patient,” or “user” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods.

A “connection” by which two components of a system, e.g., an electrical system, a data system, a computer system, a circuitry system, etc., are connected will generally be an “operable connection”, or a connection by which entities are “operably connected”. The term “operable connection” and equivalents is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, software). Logical and/or physical communication channels can be used to create an operable connection.

The term “capacitance-sensing element” refers to a structure designed to form a capacitor whose capacitance value changes in response to environmental factors such as moisture, pressure, or proximity. This may include conductive wire patterns, conductive ink traces, or other conductive materials arranged to create a measurable capacitive effect.

“NFC” or “Near Field Communication” refers to a set of communication protocols that enables two electronic devices to establish communication by bringing them within a certain proximity, typically 5 centimeters or less.

“Passive operation” refers to the ability of a device to function without an independent power source such as a battery, instead deriving necessary power from electromagnetic fields generated by an external reader device.

“Wearable substrate” refers to any flexible or semi-flexible material that can be worn on or attached to a body, clothing item, or personal accessory, and serves as the foundation for mounting or incorporating other components of the wearable device.

As used herein the terms “memory” generally refer to a physical and/or logical entity that can store data, e.g., any memory storage of a computer and is a non-transitory computer readable medium. A data store may be, for example, a database, a table, a file, a list, a queue, a heap, a memory, a register, and so on. A data store may reside in one logical and/or physical entity and/or may be distributed between multiple logical and/or physical entities. Software included in the implementation of the methods disclosed herein can be stored in the memory. The software includes, for example, firmware, one or more applications, program data, filters, rules, one or more program modules, and other executable instructions. For example, the computer can be configured to retrieve from the memory and execute, among other things, instructions related to the processes and methods described herein.

Suitable software for implementing various components of example systems and methods described herein may be developed using programming languages and tools (e.g., Java, C, C#, C++, C, SQL, APIs, SDKs, Swift, NEXTStep, SmallTalk, Unix, Objective C, assembler). Software, whether an entire system or a component of a system, may be embodied as an article of manufacture and maintained or provided as part of a computer-readable medium. Software may include signals that transmit program code to a recipient over a network or other communication medium. Thus, in one example, a computer-readable medium may be signals that represent software/firmware as it is downloaded from a server (e.g., web server).

In the foregoing description of preferred embodiments, specific terminology has been resorted to for the sake of clarity. However, the present disclosure is not intended to be limited to the specific terms so selected, and it is to be understood that each specific term includes all technical equivalents which operate in a similar manner to accomplish a similar technical purpose.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### BRIEF DESCRIPTION OF THE DRAWINGS

FIG. 1 is a picture of one embodiment of a wearable capacitance-measuring device 100 in accordance with the present disclosure, depicting a wearable substrate 120, multiple capacitance-sensing elements 140 positioned at different regions of the substrate, and an NFC component 160 (not visible).

FIG. 2A is a picture of capacitance-sensing element in the form of two generally parallel wires attached 140 to an NFC component 160, in accordance with the present disclosure.

FIG. 2B is a picture of capacitance-sensing element in the form of two generally parallel traces of conductive ink attached to an NFC component, in accordance with the present disclosure.

FIG. 3 is a picture of a partially deconstructed embodiment of a wearable capacitance-measuring device 100 in accordance with the present disclosure, depicting a wearable substrate 120, multiple capacitance-sensing elements 140 positioned at different regions of the substrate, and an NFC component 160.

FIG. 4 is a close-up picture of the connection between the capacitance-sensing element 140 and the NFC component 160 including its spiral shaped coil where current is remotely induced.

FIG. 5A is an image of an exemplary embodiment of a user interface in accordance with the present disclosure, depicting a notification 210 informing the user that the measured capacitance has exceeded a predetermined threshold.

FIG. 5B is an image of an exemplary embodiment of a user interface 200 in accordance with the present disclosure, depicting a notification 210 informing the user that the measured capacitance is below a predetermined threshold.

FIG. 6 is a graph showing measured capacitance (pF) versus water volume (ml) generated from experimental data of a wearable capacitance-measuring device in accordance with the present disclosure.

FIG. 7A is a table of experimental data used to generate the graph of FIG. 6.

FIG. 7B is a table of further experimental data used to generate the graph of FIG. 6.

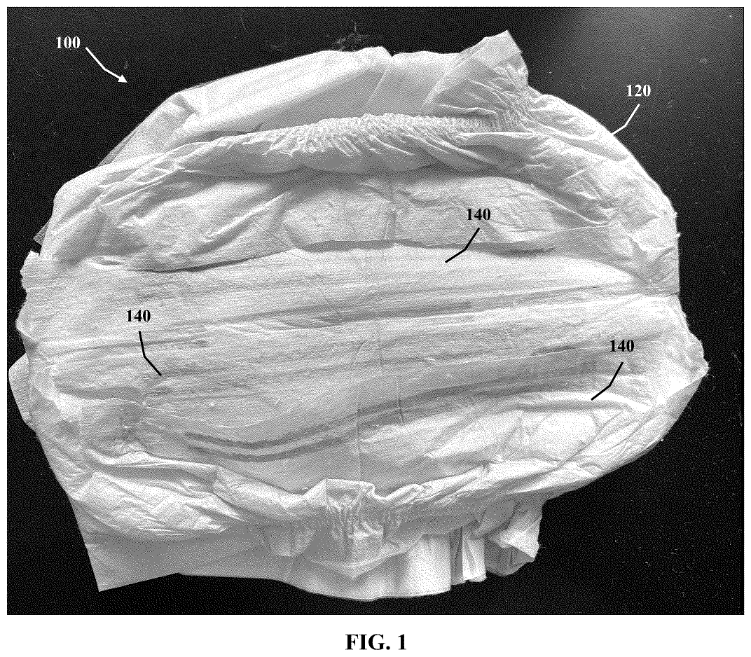

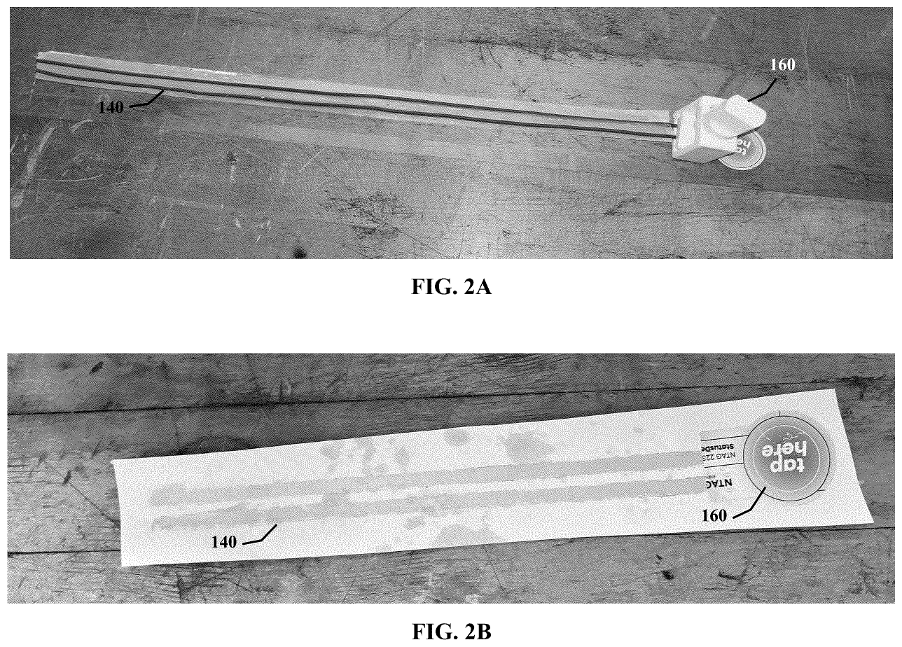

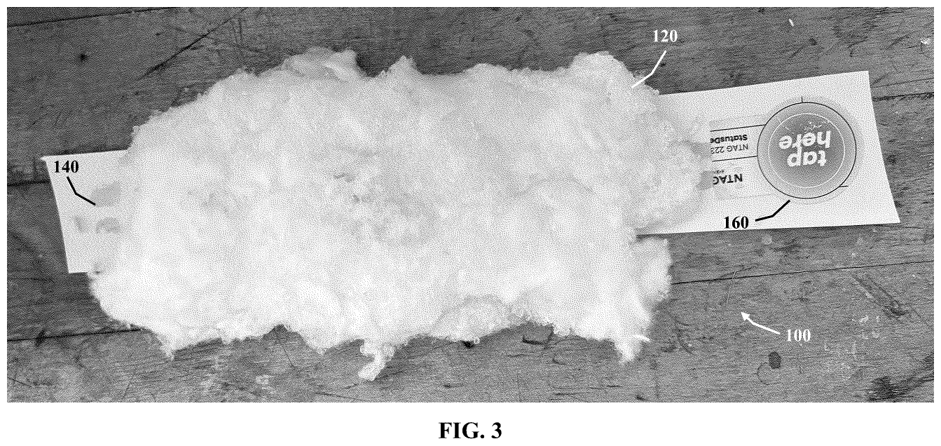

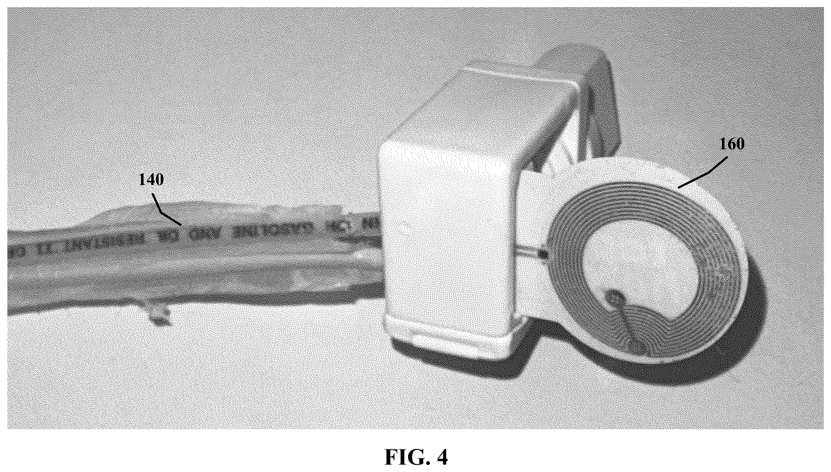

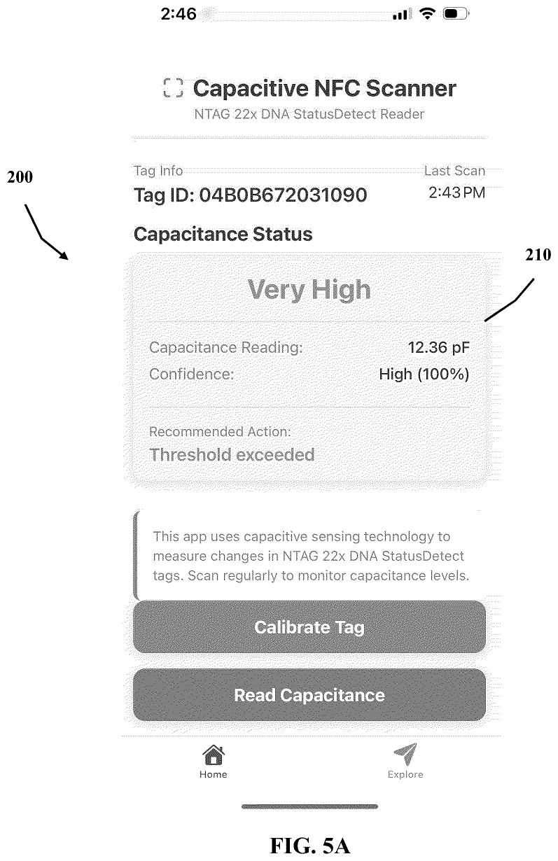

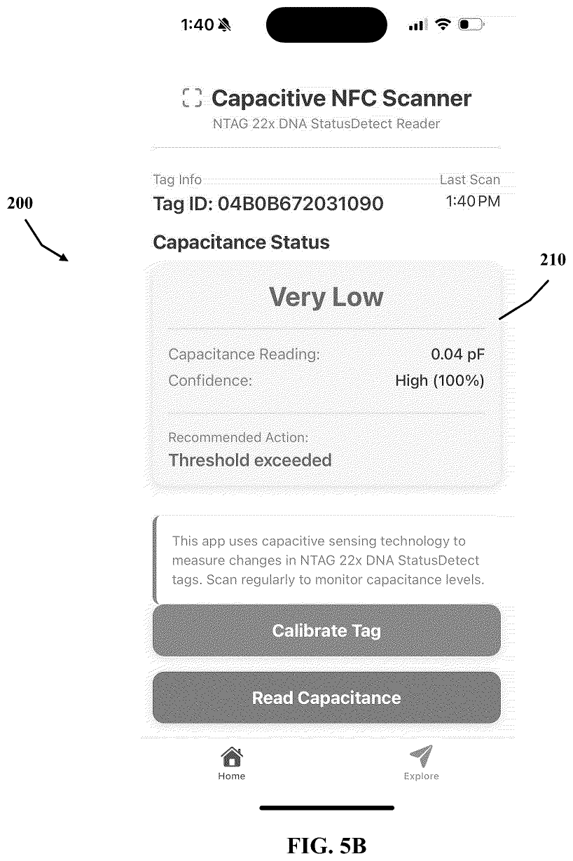

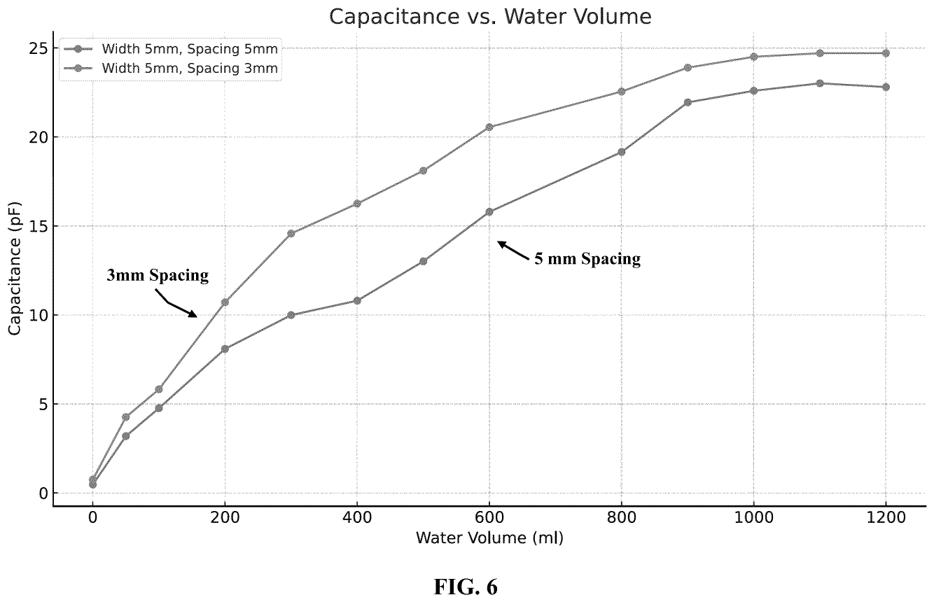

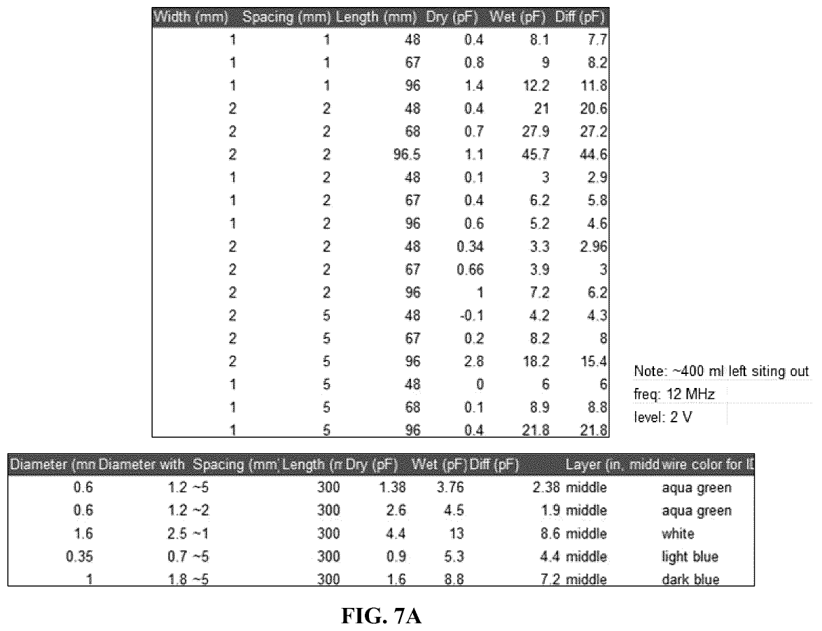

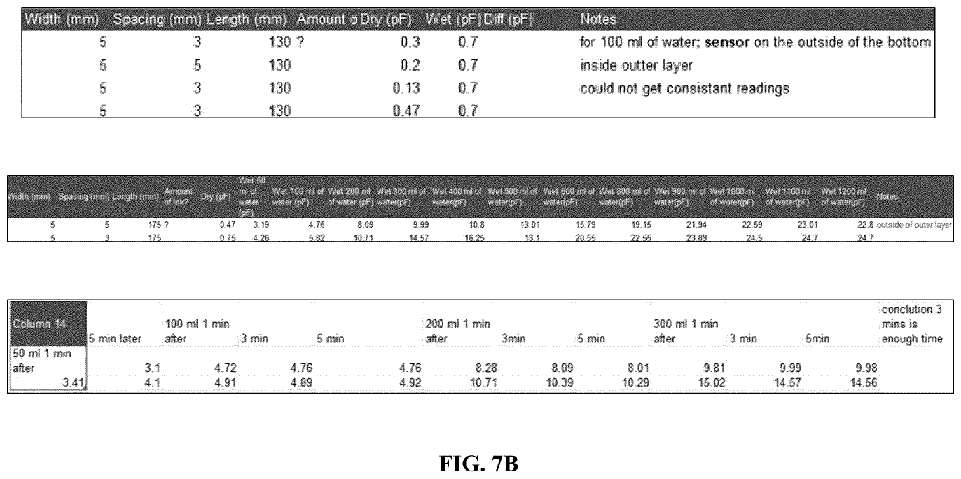

Before any embodiments are explained in detail, it is to be understood that the present disclosure is not limited in its application to the details of construction and the arrangement of components set forth in the following description or illustrated in the accompanying drawings. The present disclosure is capable of other embodiments and of being practiced or of being carried out in various ways.

### DETAILED DESCRIPTION

The following detailed description is presented to enable any person skilled in the art to make and use the disclosed subject matter. For purposes of explanation, specific details are set forth to provide a thorough understanding of the present disclosure. However, it will be apparent to one skilled in the art that these specific details are not required to practice the disclosed subject matter. Descriptions of specific embodiments are provided only as representative examples. The present disclosure is not intended to be limited to the embodiments shown, but is to be accorded the widest possible scope consistent with the principles and features disclosed herein.

The wearable capacitance-measuring device 100 with mobile application integration may comprise, in some embodiments, three primary functional components operating together: a wearable substrate 120 that serves as the base material and structural foundation, a capacitance-sensing element 140 embedded or printed onto or within the substrate 120, and an NFC component 160 connected to the capacitance-sensing element 140. Together, these components may provide passive, on-demand measurement of environmental conditions through capacitance changes, while enabling seamless integration with smartphone applications for data retrieval, interpretation, and notification. In some embodiments, additional components may be integrated with the device, such as visual indicators, supplementary passive electronic components, or multiple NFC components 160, to expand the functional capabilities of the system. The device 100 may be adapted for use across a wide variety of application domains, including, but not limited to, infant care, clinical health monitoring, athletic performance tracking, industrial environmental sensing, and consumer wellness products.

The wearable substrate 120 may serve as the primary structural foundation for the device 100, providing the flexibility, durability, and biocompatibility appropriate for wearable applications. In some embodiments, the substrate 120 may be constructed from biocompatible materials such as medical-grade fabrics, polymers, nonwoven textiles, or specialized paper-based products suitable for specific applications. The substrate 120 may vary in thickness, flexibility, and material composition depending on the intended use case, ranging from ultra-thin, breathable materials suited for health monitoring applications to more robust, multi-layered constructions appropriate for moisture detection in absorbent products. In some embodiments, the substrate 120 may incorporate multiple layers having different functional properties.

For example, the substrate 120 may include moisture-wicking layers, absorbent cores, or waterproof barrier layers that are strategically positioned relative to the capacitance-sensing element 140. In some embodiments, the substrate 120 may be formed from biodegradable or compostable materials to minimize environmental impact for disposable product applications. In other embodiments, the substrate 120 may be formed from hypoallergenic materials to enable safe and comfortable extended contact with sensitive skin. The substrate 120 may also, in some embodiments, feature a modular construction in which the capacitance-sensing element 140 and the NFC component 160 may be removably attached to the substrate 120 so that these components may be removed and reused while a disposable portion of the substrate 120 is replaced. In some embodiments, the substrate 120 may be configured as a patch, a band, a garment insert, an adhesive sticker, a diaper liner, or any other form factor suitable for positioning adjacent to a body region of interest or an environmental condition to be monitored.

The capacitance-sensing element 140 may comprise a patterned conductive material designed to form a capacitor whose electrical properties change in response to environmental factors such as moisture, pressure, or proximity. In some embodiments, the capacitance-sensing element 140 may include a pattern of conductive ink printed directly onto or within one or more layers of the substrate 120. In other embodiments, the capacitance-sensing element 140 may comprise one or more conductive wires, conductive threads, or metal foil traces arranged in a defined pattern on or within the substrate 120. The pattern of the capacitance-sensing element 140 may form interdigitated electrodes or parallel plate structures that create a measurable capacitance value. When environmental conditions change—for example, when moisture increases between or around the conductive traces, when pressure alters the distance between capacitive plates, or when a nearby object changes the effective dielectric constant of the space between the electrodes, the electrical capacitance of the element 140 changes in a measurable and reproducible manner.

This change in capacitance may be detected and quantified by the NFC component 160. The conductive pattern of the capacitance-sensing element 140 may be formed using various manufacturing techniques, including, but not limited to, screen printing, inkjet printing of silver or carbon-based conductive inks, aerosol jet deposition, vapor deposition of conductive thin films, embroidery of conductive threads, or lamination of pre-patterned conductive foil films. The specific pattern geometry, trace spacing, and electrode orientation may be optimized for different sensing applications.

For example, moisture detection embodiments may feature closely spaced interdigitated electrodes that maximize the exposed surface area in contact with a liquid analyte, while pressure detection embodiments may utilize parallel plate capacitor structures separated by a compressible dielectric layer whose thickness changes under applied force. In some embodiments, the capacitance-sensing element 140 may include multiple distinct sensing zones positioned at different regions of the substrate 120, wherein each sensing zone may independently measure a localized environmental condition. In some embodiments, one or more reference capacitor regions may be included in areas of the substrate 120 that are shielded from environmental changes, thereby providing baseline or calibration values that may be used to improve measurement accuracy and compensate for temperature-related drift.

The NFC component 160 may be electrically connected to the capacitance-sensing element 140 and may enable wireless communication with external mobile devices. In some embodiments, the NFC component 160 may comprise an integrated circuit such as an NTAG 223 DNA StatusDetect IC or a functionally equivalent NFC IC, which combines standard NFC data communication functionality with an integrated capacitance measurement circuit. The NFC component 160 may feature one or more conductive connection points that attach directly to the electrode terminals of the capacitance-sensing element 140, an integrated capacitance measurement circuit, onboard non-volatile memory for storing one or more measurement values, and an NFC antenna for wireless communication with an external reader. In some embodiments, the NFC antenna may comprise a planar spiral coil of conductive material formed on or within the substrate 120. When an NFC-enabled smartphone or other compatible reader device is brought into sufficient proximity with the NFC component 160 (typically within approximately 5 centimeters, though this distance may vary depending on antenna geometry and reader power), the alternating electromagnetic field generated by the reader induces a sufficient current in the NFC antenna to power the NFC component 160 without any battery or other stored power source in the wearable device 100. Upon receiving sufficient power, the NFC component 160 may measure the current capacitance value of the capacitance-sensing element 140, may store this measured value in its onboard memory, and may transmit the value to the external reader device via the established NFC communication link.

This passive operation may eliminate the need for batteries, rechargeable cells, or other active power sources in the wearable device 100, thereby significantly reducing the size, weight, cost, and maintenance burden of the device, while also extending its practical service life. In some embodiments, the NFC component 160 may provide additional measurement channels for independently measuring multiple capacitance-sensing zones, may provide integrated temperature measurement for environmental compensation of the capacitance readings, or may incorporate data encryption capabilities for use in privacy-sensitive healthcare applications. In some embodiments, the NFC component 160 may be compatible with NFC standards including ISO/IEC 14443, ISO/IEC 15693, or other applicable communication protocols recognized in the art.

A system for monitoring environmental conditions may include, in addition to the wearable device 100, a mobile application installed on an NFC-enabled smartphone, tablet, or other compatible mobile device. The mobile application may serve as the primary user interface for the capacitance-measuring system, providing functionality for initiating NFC scans, retrieving measurement data from the NFC component 160, interpreting the received measurements, and presenting actionable status information to the user. In some embodiments, the mobile application may include a scanning interface 200 that provides guidance to the user for properly positioning the mobile device in proximity to the NFC component 160. In some embodiments, the mobile application may include a data interpretation module that converts raw capacitance measurements received from the NFC component 160 into meaningful, human-readable status information relevant to the particular application. For example, in a moisture-sensing diaper application, the data interpretation module may categorize the received capacitance value into one or more discrete status levels, such as “dry,” “partially wet,” or “saturated,” based on predetermined or user-configured capacitance thresholds. In some embodiments, the mobile application may include a notification system configured to generate and deliver notifications 210 to the user or to one or more designated contacts, such as caregivers or healthcare providers, when the interpreted status meets or exceeds one or more predetermined threshold conditions.

Notifications may be delivered, in some embodiments, via in-app alerts, push notifications, text messages, emails, or any other communication channel supported by the mobile device. In some embodiments, the mobile application may incorporate data logging capabilities that store a timestamped record of individual measurement events, allowing users or caregivers to review trends or patterns in the monitored conditions over time. In some embodiments, the mobile application may include cloud connectivity features that allow measurement data to be synchronized with a remote server and accessed from multiple devices by multiple authorized users. In some embodiments, the mobile application may allow users to customize threshold settings and notification preferences for different individual users, different product types, or different application contexts. In some embodiments, the mobile application may incorporate machine learning or other data analysis algorithms to improve the accuracy of status interpretations based on accumulated historical data from individual users or aggregated populations.

An example embodiment of the wearable capacitance-measuring device 100 of the present disclosure may be a smart diaper system. In this configuration, the wearable substrate 120 may be incorporated into or laminated onto the absorbent layer of a diaper, with the capacitance-sensing element 140 positioned strategically to detect the presence and extent of moisture in critical regions, such as the central absorbent zone. The NFC component 160 may be embedded near the waistband or outer panel area of the diaper, where it may remain accessible for scanning while being physically separated from direct and prolonged contact with moisture. The conductive pattern of the capacitance-sensing element 140 may extend through or adjacent to the absorbent core in an interdigitated electrode configuration, whereby the closely spaced electrode traces may exhibit a pronounced change in dielectric properties when wetted. When dry, the air and dry absorbent material between the electrodes may create a relatively low baseline capacitance value. When moisture from urine or other liquid saturates the area surrounding the electrodes, the dielectric constant of the material between the electrode traces may increase significantly, resulting in a measurable and proportional increase in the capacitance of the element 140. This change may correlate with the degree of moisture saturation, allowing the mobile application to estimate relative wetness levels. The mobile application may be configured with interpretation parameters specific to diaper applications, translating capacitance measurement ranges into moisture status indicators and generating notifications to caregivers when a diaper change may be warranted.

In some embodiments, the smart diaper system may incorporate multiple sensing zones 140 at different regions of the diaper to spatially differentiate wetness distribution and identify the specific wetted area. In some embodiments, the system may generate timestamped logs of diaper change events to allow caregivers to identify patterns in voiding behavior, which may be clinically useful for infant care, elderly care, or incontinence management applications. In some embodiments, the smart diaper embodiment may incorporate a disposable/reusable hybrid design in which the NFC component 160 may be removably attached to the substrate 120 and transferred to a fresh diaper, reducing electronic waste while retaining monitoring functionality.

The device 100 and system may be implemented in various other application embodiments beyond the smart diaper example. In one such embodiment, the device 100 may be configured as a wearable health monitoring device, incorporating the capacitance-measuring technology into a wristband, adhesive skin patch, or garment-integrated sensor format. In this configuration, the capacitance-sensing element 140 may be designed to detect one or more physiological parameters associated with the user's body surface, such as perspiration levels, skin surface hydration, or subtle pressure changes associated with pulse or blood flow. The wearable substrate 120 in this embodiment may comprise breathable, medical-grade textile or polymer materials suitable for extended skin contact, while the pattern geometry of the capacitance-sensing element 140 may be optimized for the specific physiological parameter being monitored. The mobile application associated with this embodiment may include specialized algorithms for interpreting health-related capacitance changes, tracking trends over time, and optionally correlating measurements with other health metrics collected from other sensors or applications on the mobile device.

In some embodiments, additional sensing modalities may be incorporated alongside the capacitance measurement, such as temperature-sensitive elements or mechanical strain gauges, to provide supplementary data for more comprehensive health monitoring. In some embodiments, the device 100 may be configured as a clothing-integrated sensor, a headband for perspiration monitoring during exercise, a wound care monitoring patch for tracking wound exudate levels, or a specialized insole insert for pressure distribution analysis of the plantar surface of the foot. In some embodiments, the device 100 may include data security features such as encrypted NFC communication or access controls within the mobile application to protect sensitive health information in compliance with applicable privacy regulations.

In some embodiments, the system may incorporate supplementary features that enhance overall functionality and integration with other technologies or platforms. In some embodiments, the capacitance-sensing element 140 may employ specialized pattern geometries that maximize sensitivity to one specific type of environmental change while minimizing cross-sensitivity to other interfering variables. In some embodiments, the sensing structure may be multi-layered, enabling the device 100 to differentiate between different types of environmental interactions, such as distinguishing a pressure-induced capacitance change from a moisture-induced capacitance change, by analyzing the signal profiles from different electrode layers. In some embodiments, the substrate 120 may include visual indicators, such as regions coated with thermochromic or electrochromic inks, that change color or appearance in response to the same environmental conditions being electronically measured by the capacitance-sensing element 140, thereby providing a redundant visual confirmation of the condition that does not require a smartphone scan. In some embodiments, the device 100 may incorporate multiple NFC components 160, each connected to a different capacitance-sensing zone, to enable spatial mapping of the measured environmental parameter across a larger area of the substrate 120.

In some embodiments, the substrate 120 may include additional passive electronic components, such as printed resistors or printed capacitors formed by printed electronics techniques, to modify or tune the electrical characteristics of the sensing circuit for specialized frequency-response or sensitivity requirements. In some embodiments, the device 100 may be integrated directly into a manufactured product during its fabrication process, such as being incorporated as a layer within the assembly of an absorbent personal care product or embedded within the fabric of a garment, such that the sensing device is an intrinsic part of the product rather than a separately attachable add-on.

The present disclosure also provides methods of measuring environmental conditions using the wearable capacitance-measuring device 100 with mobile application integration. In one embodiment, the method may begin with applying or positioning the wearable device 100 at the intended location, such as wearing a smart wristband on a user's wrist, placing a smart diaper on an infant, or applying a wound monitoring patch over a dressing. The device 100 may remain passive and unpowered until scanned, with the capacitance-sensing element 140 exposed to the environmental conditions being monitored. When measurement information is desired, the user or caregiver may activate the mobile application on their NFC-enabled smartphone or mobile device and initiate the scanning function. The mobile application may provide guidance for proper positioning of the mobile device in proximity to the NFC component 160. When the mobile device is brought within the operational range of the NFC component 160, the NFC communication link may be established and the electromagnetic field generated by the mobile device may power the NFC component 160. Once powered, the NFC component 160 may measure the current capacitance value of the capacitance-sensing element 140, which may reflect the current environmental condition at the time of measurement. This measured capacitance value may be transmitted to the mobile application through the established NFC communication link.

The mobile application may process the received capacitance value using its interpretation algorithms, converting the raw measurement into meaningful status information relevant to the specific application, such as a diaper wetness level indicator, a skin hydration index, or a wound exudate status. The interpreted result may be displayed to the user through the user interface 200, potentially including status indicators, comparison to prior measurements, trend visualizations, or actionable recommendations. If the interpreted measurement meets or exceeds one or more predefined threshold values, the mobile application may generate one or more notifications 210 to the user or to designated contacts such as caregivers or healthcare providers. In some embodiments, the method may further include logging the measurement event with a timestamp for subsequent review and trend analysis; synchronizing measurement data with a cloud service for remote access by authorized users; correlating the capacitance measurements with other available health or environmental data for comprehensive analysis; or applying calibration adjustments based on established baseline measurements for the specific user and device combination. In some embodiments, the method may include an initial calibration step in which a baseline capacitance value is measured and stored when the device 100 is first deployed in an unused or known-dry state, providing a reference value for subsequent comparative measurements.

Various features and advantages are set forth in the following claims.

We claim:

1. A wearable device, comprising:
a substrate;
a capacitance-sensing element on or within the substrate, wherein the capacitance-sensing element changes capacitance in response to environmental conditions; and
an NFC component connected to the capacitance-sensing element, configured to measure the capacitance when activated by an external device.

2. The device of claim 1, wherein the environmental conditions comprise at least one of moisture, pressure, or proximity.

3. The device of claim 1, wherein the capacitance-sensing element comprises a conductive material arranged in a pattern.

4. The device of claim 3, wherein the conductive material comprises at least one of conductive ink, conductive wire, conductive thread, or metal foil.

5. The device of claim 3, wherein the pattern comprises interdigitated electrodes.

6. The device of claim 1, wherein the NFC component operates without a battery.

7. The device of claim 1, wherein the substrate is incorporated into at least one of a personal care product, a wearable accessory, or clothing.

8. The device of claim 1, wherein the substrate comprises a biocompatible material.

9. The device of claim 1, wherein the capacitance-sensing element comprises a plurality of sensing zones positioned at different regions of the substrate, wherein each sensing zone is configured to independently measure a localized environmental condition.

10. The device of claim 1, wherein the NFC component comprises an NFC antenna and an integrated circuit comprising a capacitance measurement circuit and onboard memory configured to store at least one measured capacitance value.

11. A system for monitoring environmental conditions, comprising:
a wearable device comprising a substrate, a capacitance-sensing element on or within the substrate, and an NFC component connected to the capacitance-sensing element; and
a mobile application on a mobile device configured to activate the NFC component, receive capacitance data from the NFC component, and determine a status based on the capacitance data.

12. The system of claim 11, wherein the mobile application is further configured to generate a notification based on the determined status.

13. The system of claim 11, wherein the wearable device operates without a battery.

14. The system of claim 11, wherein the mobile application is further configured to store historical data from a plurality of measurements and display a trend based on the historical data.

15. The system of claim 11, wherein the mobile application is further configured to synchronize the capacitance data with a remote server and provide access to the data from a plurality of authorized user devices.

16. The system of claim 11, wherein the wearable device is incorporated into a personal care product, and wherein the mobile application is configured to interpret the capacitance data to determine a moisture status of the personal care product.

17. A method of measuring environmental conditions, comprising:
providing a wearable device comprising a substrate, a capacitance-sensing element on or within the substrate, and an NFC component connected to the capacitance-sensing element;
bringing a mobile device into proximity with the wearable device such that the mobile device activates the NFC component via an electromagnetic field;
measuring, by the NFC component, a capacitance value of the capacitance-sensing element;
transmitting the capacitance value to a mobile application on the mobile device; and
determining, by the mobile application, a status based on the capacitance value.

18. The method of claim 17, further comprising generating, by the mobile application, a notification based on the determined status.

19. The method of claim 17, further comprising storing the capacitance value with a timestamp to create a measurement log, and displaying, by the mobile application, trend information derived from a plurality of stored measurements.

20. The method of claim 17, further comprising performing a calibration measurement of the capacitance-sensing element under a known environmental condition, and applying the calibration measurement as a baseline value when determining the status.
